(19) United States
(12) Patent Application Publication
(10) Pub. No.: US 2009/0306359 A1
(43) Pub. Date: Dec. 10, 2009

(54) NON-ALCOHOLIC BUFFER FORMULATIONS FOR ISOLATING, PURIFYING AND RECOVERING LONG-CHAIN AND SHORT-CHAIN NUCLEIC ACIDS

(76) Inventors: Timo Hillebrand, Honow (DE); Peter Bendzko, Berlin (DE)

Correspondence Address: BUCHANAN INGERSOLL & ROONEY PC, P.O. BOX 1404, ALEXANDRIA, VA, 22313-1404, US

(21) Appl. No.: 12/408,015

(22) Filed: Mar. 20, 2009

Related U.S. Application Data
(63) Continuation-in-part of application No. 10/534,387, filed on Nov. 22, 2005, now abandoned.

(30) Foreign Application Priority Data
Nov. 8, 2002 (DE) 102 52 545.5
Nov. 14, 2002 (DE) 102 53 351.2

(51) Int. Cl. C07H 21/00

(52) U.S. Cl. 536/25.41

(57) ABSTRACT

Embodiments relate to methods and formulations of buffers used for isolating, purifying, and recovering long-chain and short-chain nucleic acids. The areas of application of the inventive method include all laboratories engaged in isolating nucleic acids. In one embodiment a solution containing a nucleic acid is prepared with additives containing monovalent and multivalent cations and, optionally, an alcohol and/or additional additives. The solution is contacted with a solid phase, the solid phase is optionally washed, and the nucleic acid is removed. The solution may contain multivalent and/or monovalent cations and may contain an alcohol. The solution in certain embodiments has a pH between 7 and 10. Ammonium chloride, sodium chloride and/or potassium chloride may be used as monovalent salt components. Magnesium chloride, calcium chloride, zinc chloride and/or manganese chloride may be used as multivalent salt components. In a preferred embodiment, identical molar amounts of sodium chloride and manganese chloride are used.

### CROSS-REFERENCE TO RELATED APPLICATIONS

This application is a continuation-in-part of and claims priority to pending U.S. patent application Ser. No. 10/534,387, which was the United States national phase application under 35 U.S.C. 371 of International Application No. PCT/DE2003/003728, which was filed on Nov. 10, 2003, and which claimed priority to German Application No. 102 52 545.5, filed Nov. 8, 2002, and German Application No. 102 53 351.2, filed on Nov. 14, 2002. All of the foregoing are incorporated by reference herein.

### BACKGROUND OF THE INVENTION

1. Field of the Invention

The method's areas of application include all laboratories engaged in isolating nucleic acids, such as those dealing with forensic medicine, food diagnosis, medical diagnosis, molecular biology, biochemistry, genetic engineering, and all other related fields.

2. Background of the Art

Under classic conditions, DNA is isolated from cells and tissue whereby the base materials are dissolved under highly denaturing and reducing conditions, partially also through the use of protein-digesting enzymes. The emerging nucleic acid fraction is cleaned by means of phenol-/chloroform extraction steps, and the nucleic acids are obtained through dialysis or ethanol precipitation from the aqueous phase (Sambrook, J., Fritsch, E. F. and Maniatis, T., 1989, CSH, “Molecular Cloning”).

These “classic procedures” to isolate nucleic acids from cells and especially from tissue are very time-consuming (sometimes longer than 48 hours), require a considerable amount of sophisticated equipment, and can also not be carried out in field conditions. Besides, such methods pose a not insignificant health hazard due to the use of chemicals such as phenol and chloroform.

Various alternative procedures to isolate nucleic acids from different biological base materials allow one to circumvent the time-consuming and toxic phenol-/chloroform extraction from nucleic acids and reduce the time spent in carrying out the process.

All these procedures are based on a method developed and first described by Vogelstein and Gillespie (Proc. Nat'l Acad. Sci., US, 1979, 76, 615-619) regarding the preparative and analytic purification of DNA fragments from agarose gels. The method combines the dissolution of the agarose containing the DNA strand to be isolated in a saturated solution of chaotropic salt (NaI) with a binding of the DNA on glass particles. The DNA affixed to the glass particles is then washed with a washing solution (20 mM tris-HCl [pH 7.2]; 200 mM NaCl; 2 mM EDTA; 50% v/v ethanol) and finally removed from the carrier particles.

Until now, this method experienced a series of modifications and is currently used for various procedures pertaining to the extraction and purification of nucleic acids from diverse sources (Marko, M. A., Chipperfield, R. and Bimboim, H. G., 1982, Anal. Biochem., 121, 382-387).

In addition, there are currently numerous reagent systems worldwide, especially for the purification of DNA fragments from aragose gels and the isolation of DNA plasmid from bacterial lysates, as well as for the isolation of long-chain nucleic acids (genomic DNA, cellular total RNA) from blood, tissue, or also cell cultures.

All these commercially available kits are based on the conventionally known principle of nucleic acids binding on mineral carriers given the presence of solutions of various chaotropic salts and using suspensions of finely ground glass powder (e.g. Glasmilk, BIO 101, La Jolla, Calif.), diatomaceous earth (Sigma) or also silica gels (Diagen, DE 41 39 664 A1).

A procedure to isolate nucleic acids practical for many different applications is portrayed in U.S. Pat. No. 5,234,809 (to Boom). It describes a process to isolate nucleic acids from nucleic acid-containing base materials through the incubation of the base material with a chaotropic buffer and a DNA-binding solid phase. The chaotropic buffers carry out both the lysis of the base material as well as the binding of the nucleic acids to the fixed phase. The process is well suited to isolate nucleic acids from small sample quantities and in particular it has a practical application in the field of isolating viral nucleic acids.

Significant disadvantages of the process are, among other things, that the dissolving carried out by the chaotropic buffers is not usable for all materials, or is only extremely inefficient for larger quantities of base materials and can only be completed in a very time-consuming way. In addition, mechanical homogenization procedures are required, if for example DNA is to be isolated from tissue samples. Furthermore, different problems require the use of various chaotropic buffers of continuously different concentrations. The process is thus in no way universally usable.

The physical-chemical principle of today's commercially available systems, according to known prior art, used to isolate nucleic acids based on the binding of nucleic acids to the surfaces of mineral carriers consists of disturbing the superordinate structures of the aqueous environment through which the nucleic acids adsorb on the surface of mineral materials, especially glass or silica particles. The disturbance of the superordinate structures of the aqueous environment always results under the presence of chaotropic ions and is almost quantitative at high concentrations of these. Given this described physical-chemical basis, all commercially available systems for the isolation of nucleic acids contain buffer compounds with highly ionic chaotropic salts for the binding of nucleic acids to a nucleic acid binding solid phase.

Specific modifications of these processes pertain to the use of specific carrier materials that exhibit application-related advantages for certain tasks (see, for example, Invitek GmbH WO 95/34569), yet have the same disadvantages.

A commonality of all the described processes for the isolation of nucleic acids by means of binding the nucleic acids to mineral solid phases using chaotropic saline solutions is that for the binding of nucleic acids to the selected carrier materials, high concentrations of carrier materials must be utilized. Such chaotropic salts (e.g. guanidinisothiocyanate, guanidine hydrochloride, sodium perchlorate or sodium iodide) are highly toxic substances. The highly ionic buffer systems utilized often result in a transfer of salt contamination into the nucleic acids to be isolated and are often the reason that a series of downstream applications (PCR, restricted digestion, hybridization, and ligations) cannot be carried out and if so only partially. In addition, when dealing with chaotropic buffers there is a considerable health hazard (especially with long-term exposure) as well as substantial environmental damage due to sewage-borne pollutants.

Patent DE 198 56 064 describes a first-ever innovative process to isolate nucleic acids. According to it, the binding of the nucleic acids to be isolated to mineral carriers results without the use of previously required highly ionic chaotropic salts. The process is based (as are the chaotropic processes) on the lysis of the base material, the binding of the nucleic acid to a mineral carrier material, the subsequent washing of the bound nucleic acids with ethanol-containing washing buffers, the removal of ethanol and the final elution of the nucleic acids with a weakly ionic elution buffer or water.

For special protocols, e.g. the isolation of PCR fragments from amplification formulas, the lysis step is not required. The PCR reagent formula is mixed with a required binding buffer and incubated with a mineral carrier material. There then follows washing with an ethanol-containing washing buffer, then the removal of ethanol, and finally the elution of the bound nucleic acid from the carrier material. That the processes without the use of chaotropic buffers have obvious advantages is demonstrated by the fact that after this initial description, other patents (e.g. DE 100 33 991) describe the potential advantages of this new binding chemistry.

Interestingly, it appears that all commercially available systems worldwide for the isolation of nucleic acids based upon the binding of nucleic acids to mineral carrier materials (magnetic particles, membranes, carrier suspensions, among others) operate primarily according to the described process. Since the initial description by Vogelstein and Gillespie, the bound nucleic acids are always washed with alcohol or acetone-containing saline solutions. The washing steps are an essential component of the extraction protocol and besides the removal of bound undesirable inhibitory materials, they also always serve in the necessary removal of the salts required for the binding of the nucleic acids.

However, the use of alcohol or acetone-containing washing buffers always signifies a quite substantial disadvantage unsolved to date. It is known that even traces of alcohol in the final nucleic acid can substantially impair downstream applications. For that reason, it is always necessary to integrate an ethanol-removal step in the actual process to isolate or purify nucleic acids.

This is always problematic when magnetic particles or particular carrier suspensions are used, the process requires considerable time, and can result in the irreversible loss of nucleic acids, especially in the over-drying of carrier materials.

The step of removing residual alcohol is especially problematic for applications that isolate nucleic acids in high throughput ranges. Generally, up to 30 minutes are required within the scope of automated nucleic acid purifying processes to free membranes in filter disks or magnetic particles of the residual alcohol. In addition, the actual washing steps with alcoholic wash buffers are very time consuming and obviously also cost-intensive especially in high throughput ranges.

From the disadvantages of the prior art, the task emerges to avoid the use of alcoholic components and to thereby achieve a substantial shortening of the isolation and purification process.

### BRIEF SUMMARY OF THE INVENTION

Embodiments of the invention relate to novel formulations of buffers for isolating, purifying, and recovering long- and short-chain nucleic acids. Preferred embodiments exclude alcohol from the washing mixture used to remove the separated nucleic acids from the support. Particularly preferred embodiments further exclude alcohol from the binding mixture.

### BRIEF DESCRIPTION OF THE FIGURES

FIG. 1 shows purification of a range of DNA fragments from an aqueous solution and comparison with various binding buffers in regard to binding efficiency.

FIGS. 2 through 5 show removal of impurities resulting in production of high-purity DNA.

FIG. 6 shows cut DNA fragments discussed in Example 4.

FIG. 7 shows the effects of a washing procedure reported in Example 5.

FIG. 8 shows the effects of a washing procedure report in Example 5.

### DETAILED DESCRIPTION OF THE INVENTION

Based on the use of non-chaotropic buffer formulas, as already described in patent DE 198 56 064, one can see that only unexpectedly low concentrations are required for the binding of nucleic acids to be isolated.

The invention's core point is in the simultaneous use of mono- and multivalent cations, preferably divalent cations, for the binding of nucleic acids to the solid phase.

Na⁺, K⁺ and NH⁺ in the form of the corresponding salts are preferred as monovalent cations. Mg⁺², Ca⁺², Zn⁺² and Mn⁺² in the form of corresponding salts are preferred as divalent cations. An especially preferred embodiment is the combined use of Na⁺ and Mg⁺².

According to the invention, the mono- and multivalent cations, preferably divalent cations, can be used in diverse quantity ratios. Successful results are best achieved in the broad range of mono-/divalent cations in a molar ratio of 9:1 to 1:9. Ratios of 7:3 to 3:7 and 6:4 to 4:6 are preferred. Especially preferred are the embodiments with identical (1:1) or almost identical molar masses of mono- and divalent cations. The total cation concentration in the solution prior to binding to the solid phase is preferably <0.5M.

If the nucleic acids are present in a solution that already contains mono- or divalent cations, e.g. after a preceding lysis of diverse base materials, then the present amount of cations is taken into account in adjusting the optimal cation concentration according to the invention. Thus, if the lysis buffer contains divalent cations that will be contained in the solution after lysis, then only the required amount of monovalent cations is added (or vice versa).

An especially important characteristic of the invention is also that the washing buffers used according to the invention contain no alcoholic components, as was the case in all other prior art processes.

Surprisingly, in the combination of salts of a monovalent cation with a multivalent one that lie in a concentration range required for binding, the buffer formulations consisting of only one salt each are no longer sufficient for the binding to occur. Thus, the combination of magnesium chloride and sodium chloride at quantities of less than 5 mM each allow the binding of nucleic acids of an additional magnitude spectrum (Example 1). The use of mixed salts having monovalent and multivalent cations as integral components of binding buffers for the binding of nucleic acids to mineral carriers has not been described to date. The surprising result now allows an entirely new strategy to isolate and purify nucleic acids from complex base materials. Since surprisingly only extremely low salt concentrations are required in binding buffers, it becomes possible to isolate nucleic acids from complex samples or solutions that contain a plurality of materials to be removed with innovative washing buffers without the previously required ethanol or also entirely without a washing step.

This has tremendous advantages for the isolation of nucleic acids and solves the described problems of using alcohol-containing washing buffers, especially for automated high throughput applications, in an ideal manner. Thus, the use of buffer formulations according to the invention allows, for example, the purification of PCR products made of complex PCR reagent formulas for a subsequently sensitive sequencing reaction and without a single washing step in the form of an automated application (binding of the PCR products to the filter membrane of a 96-gauge corrugated plate) in less than 10 minutes. Previous procedures based on the binding of nucleic acids to a solid phase require about 45 minutes to 1 hour. In addition, the process sequence is also extremely simple and only consists of mixing the PCR starting solution with a binding buffer according to the invention, the transfer of the solution to the filter plate, the suctioning through of the solution, and the subsequent elution of the PCR products using water or a 10 mM tris-buffered aqueous solution. Thus, PCR products can be purified in an extremely timesaving, harmless, and cost-effective manner. The throughput can therefore be increased dramatically (also for less sophisticated equipment; e.g. a robot no longer requires a washing tool). The quality of the purified PCR products is very high, which one can see in the clean sequence reactions (Example 2).

Furthermore, surprisingly, one can see that nucleic acids having excellent quality and quantity can also be isolated from complex biological samples without washing steps or using an alcohol-less washing buffer. The isolation of a DNA plasmid described in an example in unexamined patent DE 100 33 991 without a washing step did not result from the previously manufactured, clarified lysate. The DNA plasmid to be isolated was first produced in a pure form by means of known standard procedures and this DNA plasmid was again incubated with a buffer, bound to a solid phase, and then after the necessary removal of the binding buffer's alcohol re-released from the solid phase.

With embodiments of this invention, it is now possible to purify DNA plasmid directly from the clarified lysate, whereby again no washing with an alcohol-containing washing buffer is necessary, or the DNA plasmid can be isolated without a washing step directly after the resulting binding to a solid phase. The DNA plasmid is thus again of excellent quality and quantity (Example 3).

Embodiments of the invention also allow one to isolate genomic nucleic acids from complex biological samples in an extremely fast, cost-effective and simple manner. Accordingly, only a base material using for example a traditional protease K-digestion is dissolved by standard means in a buffer compatible for this purpose, then the lysed sample is mixed with a non-chaotropic binding buffer according to this invention, and the solution is incubated with a nucleic acid-binding solid phase, and if necessary washed with a non-alcoholic washing buffer (or if required, without a washing step) and then the genomic nucleic acid is isolated from the solid phase using water or a tris-solution. The quality of the isolated DNA is again very high and can be used immediately for further applications.

Another variant of this invention results from the observation that the pH value of the binding buffer used has considerable influence on the yield of the nucleic acid to be produced and it possesses a selectivity in regard to the fragment lengths of the PCR products to be purified for example. It is not necessary to combine mono- and multivalent salts with each other in a solution. Divalent salts are preferred, and Mg or Ca salts are especially preferred for use.

This allows one to exclude selecting the pH value of binding buffers, e.g. small PCR products, from the isolation process. In addition, one can also see that surprising effects are achieved through the combination of salts and alcohol in the binding buffer in relation with the selection of the pH value. The alcoholic components in the binding buffer enable selectivity in regard to the size-related percentage of DNA fragments. The tendency is for a reduction of the binding buffer's pH value to reduce the yield of smaller DNA fragments up to complete inhibition of recovery. This is especially significant when heterogeneous sample mixtures are present.

Thus, for pH values >8, an almost quantitative recovery of DNA fragments from a broad size range (100 bp to 10,000 bp) can be achieved with binding buffers, if the binding buffer contains an alcoholic component.

For alcohol-containing binding buffers the pH value is set to 5-9.5 and preferably to 8-9.5, 6.5-8 or 5-6.5. Thus the recovery of certain fragment sizes is possible, i.e. certain fragment sizes are not recovered. It should be pointed out that the wording “no recovery” is not meant in the absolute sense, i.e. traces of nucleic acid fragments could always be recovered in a non-specific manner.

For alcohol-less binding buffers, recovery of DNA fragments results in quantitative amounts and over of a size range of 100 bp to 10,000 bp preferably at a pH value of >8.5. Finally, this invention allows one to develop binding buffers by means of combining salt components and alcohol components as well as modifying the pH value, which allow a selective recovery of certain nucleic acid fragments from heterogeneous base samples.

Embodiments of this invention thus permit a clear simplification of the process to isolate nucleic acids from complex nucleic acid-containing samples. The process no longer requires toxic chemicals; the salt quantities used are dramatically reduced and clearly results in a less toxic impact on the environment; and the processes require less steps and are therefore significantly faster than previously used methods. Especially for high throughputs, there are now cost-effective and extremely fast processes available. The composition of the buffer formula also enables one to achieve a selective size-related percentage of DNA fragments to be recovered.

Other embodiments of the invention contemplate a binding mixture that excludes alcohol. Surprisingly, such a mixture works not only for washing of bound DNA as described in experiment 1 of Example 5, it also worked directly for binding, as shown in experiment 2 of Example 4. Use of the alcohol-free mixture eliminated the need for a washing step.

The alcohol-free binding mixture was found to work in a pH range between 8.5 to 9.5. Those skilled in the art would also recognize that effective pH ranges would be between 9.0 to 9.5, and 8.5 to 9.0. In the case of keeping this pH range, a system with optional washing steps was created without any alcohol in binding mixture or in the optional wash buffer. Again, chaotropic salts may be excluded.

In the case of broader pH range (pH 5.5-9.5) a system with optional washing steps was created containing alcohol in binding mixture and optional wash buffer. As published in United States Patent Application Publication No. 20060160085 (U.S. Ser. No. 10/534,387), incorporated by reference herein, also the procedure works in the case of a binding mixture with alcohol and an optional washing mixture without alcohol.

The possibility to do the complete procedure without alcohol shortens the protocol efficiently, using alcohol in such mixtures requires a drying step of 5-20 minutes depending on the protocol. Also, alcohols are cost-intensive parts of such mixtures, and their flammability increases problems in their use and transportation.

Embodiments of the invention are explained below using examples. The examples are not intended to represent any limitation to the claims, unless such limitation is specifically stated in the claims.

### EXAMPLES

### Example 1

Purification of a range of DNA fragments from an aqueous solution; comparison of various binding buffers in regard to binding efficiency.

TH1 buffer (5 mM NaCl; 5 mM MgCl₂/tris HCl, isopropanol)
TH2 buffer (10 mM MgCl₂/tris HCl, isopropanol)
TH3 buffer (10 mM NaCl/tris HCl, isopropanol)

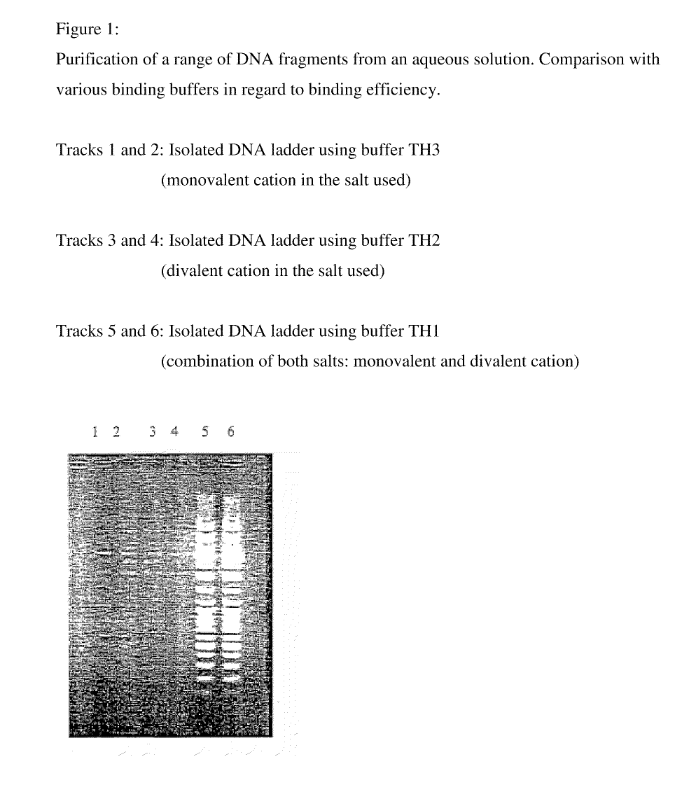

The TH1 buffer is a combination of a monovalent and divalent salt at a total strength of 10 mM. Buffers TH2 and TH3 each contain only one salt (with a monovalent cation and a divalent cation, respectively) for a total strength of 10 mM. 130 μl of the respective buffers were mixed with an aqueous 50-μl solution containing a commercial DNA ladder (Fermentas) and the solution is transferred to a centrifuge stand with a glass fiber fleece. Then, the solution was centrifuged for 1 minute at 10,000 rpm and the centrifuge column was transferred to a new reaction vessel. The elution of the bound fragments results by adding 30 μl of a 10 mM tris-HCl solution and subsequent centrifuging for 1 minute. The total time to isolate the DNA fragments was thus only about 2 minutes. The eluates obtained were applied to a 1.2% agarose gel and electrophoretically cut. As can clearly be seen in the electrophoretic image, efficient recovery of DNA fragments results using only the combination buffer. The single-salt containing binding buffers however only show a very slight binding ability (FIG. 1).

### Example 2

Purification of PCR products from a complex PCR reagent solution and subsequent use of the purified PCR products for DNA sequencing.

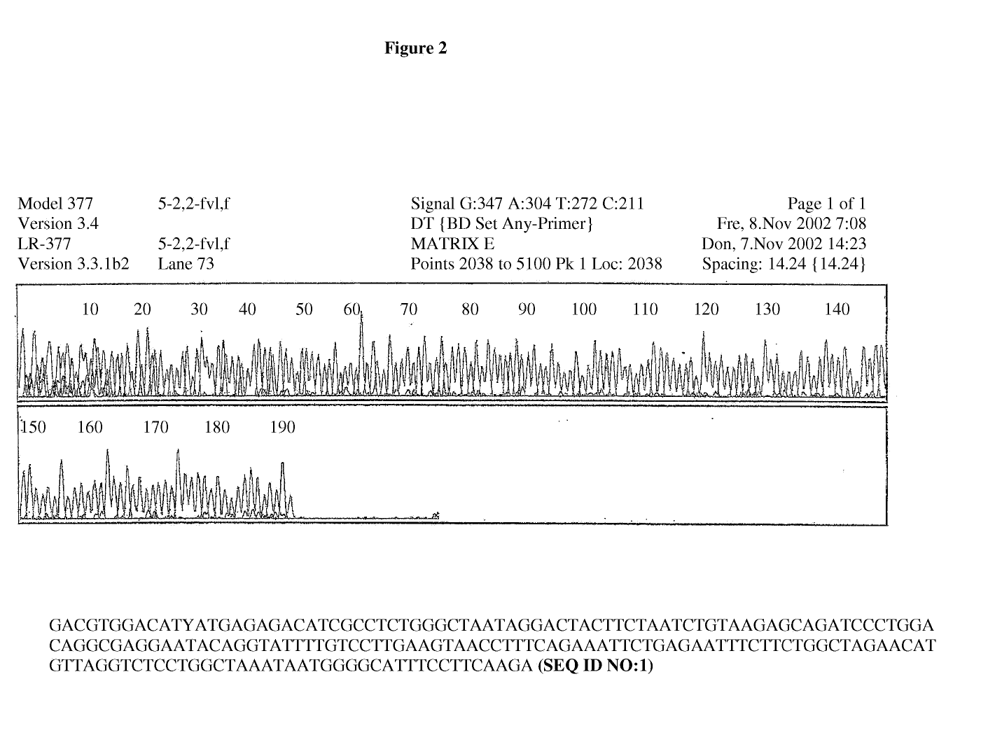

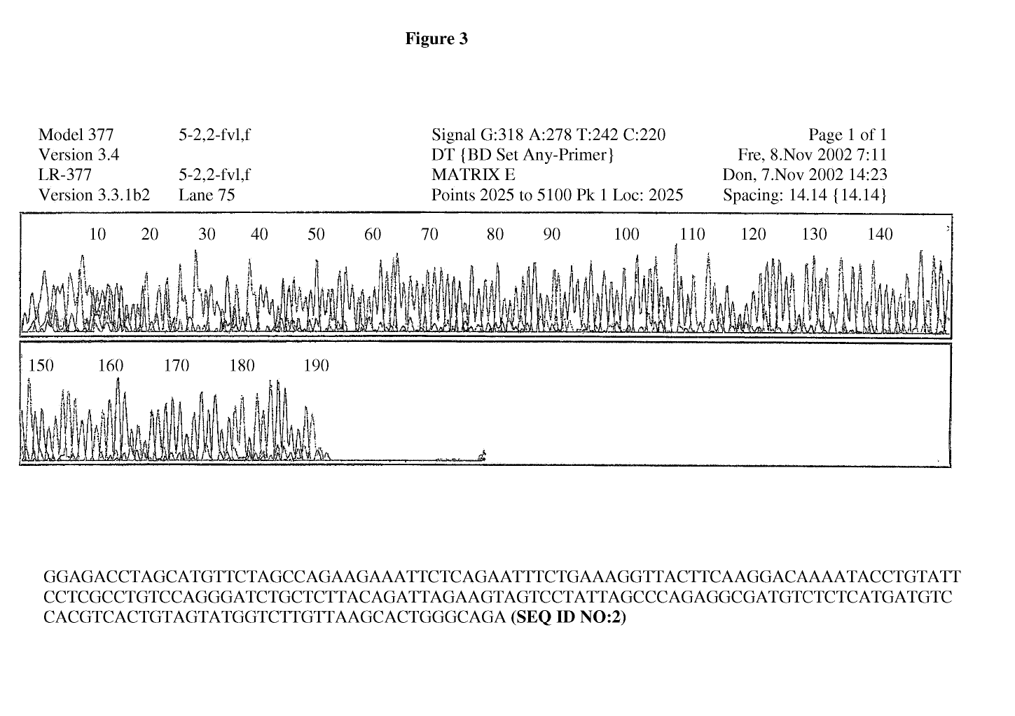

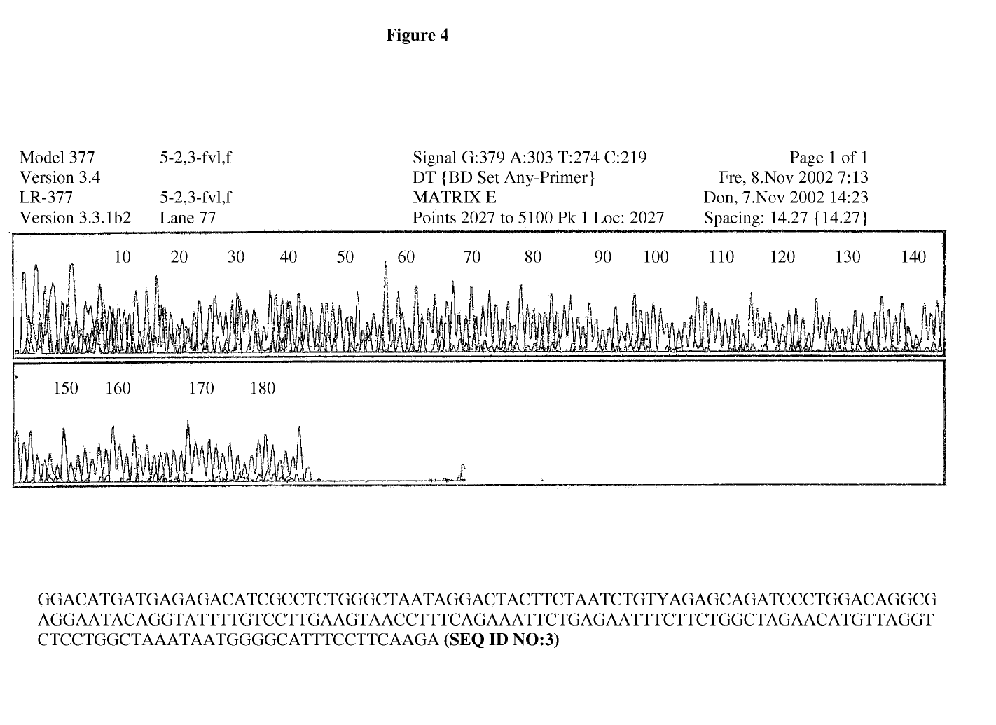

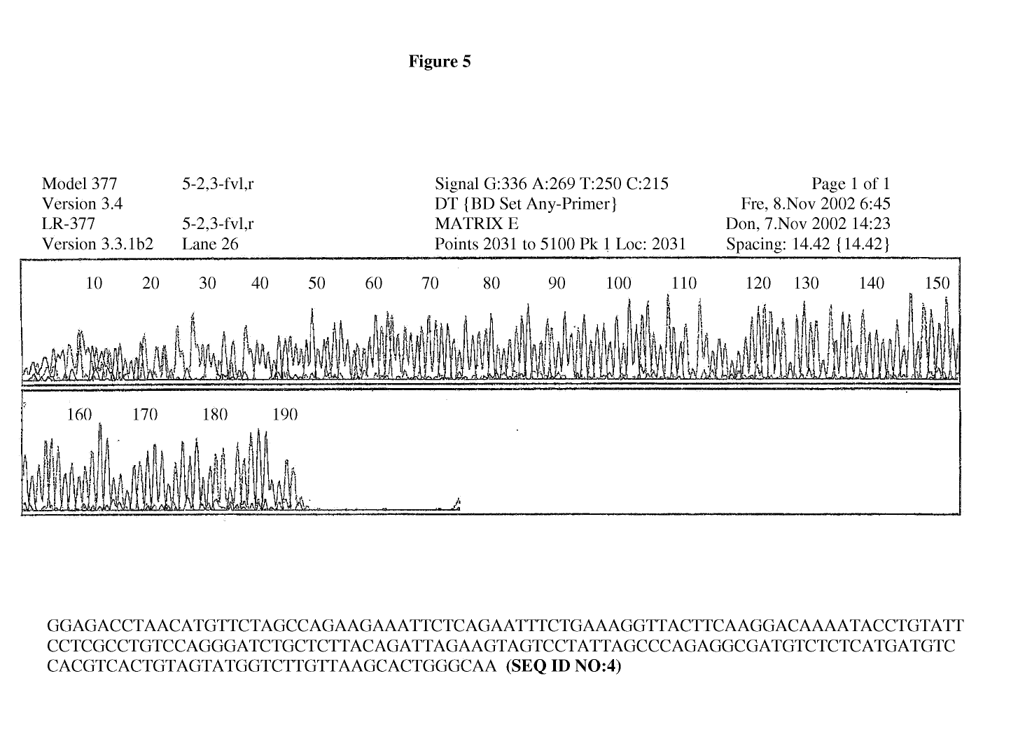

50 μl PCR reagent solutions were mixed with 130 μl of TH1 and TH4 binding buffers (50 mM NaCl; 50 mM MgCl2/tris HCl, isopropanol), then transferred to a centrifuge column with glass fiber fleece, centrifuged for 1 minute and finally the DNA is eluated from the column using 10 mM of tris HCl. The isolated PCR products were then used for the sequencing. The sequencing results confirm that without using previously required wash steps, all interfering components were efficiently removed and high-purity DNA results (FIGS. 2-5).

### Example 3

Isolation of DNA plasmids from bacterial lysates. Comparison of the purity of the isolated DNA plasmids for various washing conditions or without a washing step.

2 ml of a bacterial overnight culture (XL-1 with pGEM plasmid) were centrifuged and the pellet was re-suspended with 200 μl of solution I (tris, EDTA Rnase A). Then, 200 μl of solution II (SDS/NaOH) were added. The reaction vessels were shaken carefully several times. Then 200 μl of a solution III (250 mM MgCl2/tris HCl) were added. The reaction vessels were carefully and briefly shaken and centrifuged for 5 minutes at maximum rpm. The clarified excess was mixed with 100 μl isopropanol and given to a centrifuge column with a glass fiber fleece and centrifuged for 1 minute. Then, each of the 3 samples was immediately mixed with 10 mM tris (no washing). 3 samples were mixed with 800 μl of an alcohol-less washing buffer (10 mM NaCl/10 mM MgCl2/tris HCl) and centrifuged for 1 minute and then the DNA plasmid was eluted with 10 mM tris-HCl from the column and 3 samples were washed with a 70% solution of ethanol. Then the ethanol was removed and the DNA plasmid was eluted again by adding 10 mM tris-HCl.

The following table illustrates the required preparation time, the quality and quantity of the isolated DNA plasmid.

| Sample | Washing step | Ration 260:280 | Yield | Preparation time |
|---|---|---|---|---|
| 1 | No washing | 1.71 | 12 μg | 8 min |
| 2 | No washing | 1.74 | 15 μg | 8 min |
| 3 | No washing | 1.81 | 14 μg | 8 min |
| 4 | Washing w/o | 1.82 | 15 μg | 10 min |
| | alcohol | | | |
| 5 | Washing w/o | 1.81 | 14 μg | 10 min |
| | alcohol | | | |
| 6 | Washing w/o | 1.84 | 12 μg | 10 min |
| | alcohol | | | |
| 7 | Washing w/ | 1.86 | 14 μg | 16 min |
| | alcohol | | | |
| 8 | Washing w/ | 1.92 | 16 μg | 16 min |
| | alcohol | | | |
| 9 | Washing w/ | 1.89 | 13 μg | 16 min |
| | alcohol | | | |

### Example 4

Purification of a range of DNA fragments from an aqueous solution; comparison of various binding buffers in regard to binding efficiency in relation to pH value of the binding buffer.

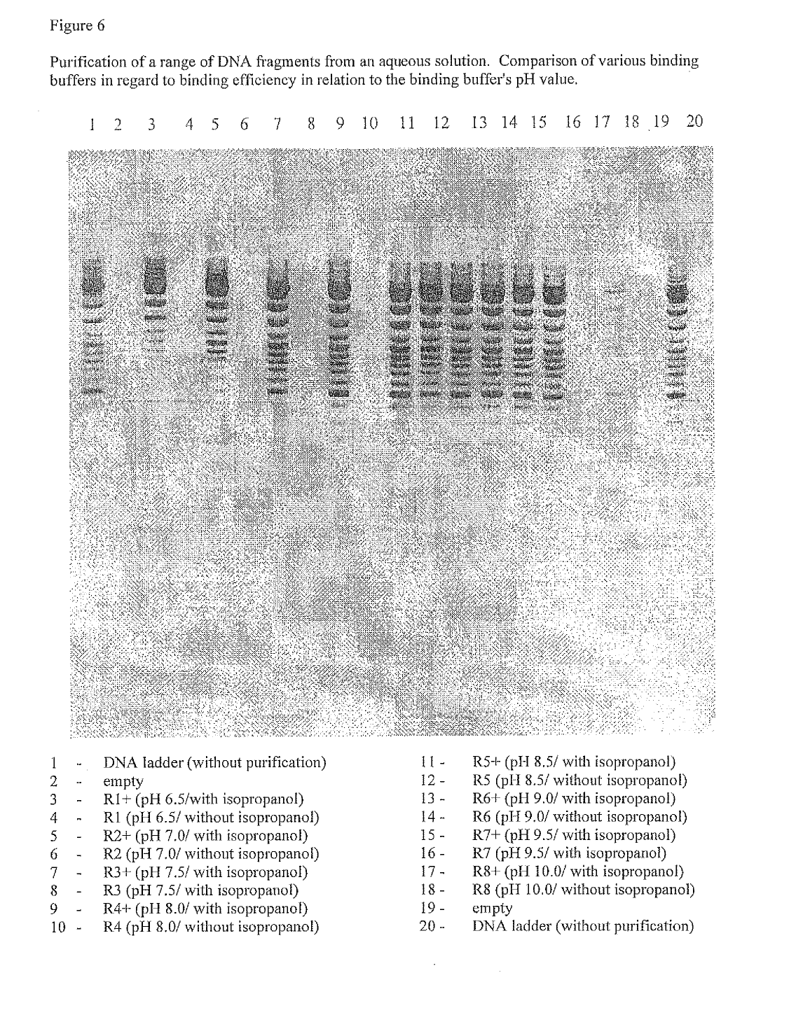

50 μl of an aqueous solution, containing 2 μg of a DNA ladder (Fermentas) were mixed with 130 μl of binding buffer (R1-R8), then transferred to a centrifuge column with a glass fiber fleece, centrifuged for 1 minute and finally the DNA was eluted from the column using 10 mM of tris-HCl. The isolated DNA fragments were then electrophoretically cut (FIG. 6).

Binding buffer: each containing 50 mM MgCl2 and optionally 20% isopropanol as well as 100 mM tris HCl with varying pH values.

R1+ (pH 6.5/with isopropanol)
R1 (pH 6.5/without isopropanol)
R2+ (pH 7.0/with isopropanol)
R2 (pH 7.0/without isopropanol)
R3+ (pH 7.5/with isopropanol)
R3 (pH 7.5/without isopropanol)
R4+ (pH 8.0/with isopropanol)
R4 (pH 8.0/without isopropanol)
R5+ (pH 8.5/with isopropanol)
R5 (pH 8.5/without isopropanol)
R6+ (pH 9.0/with isopropanol)
R6 (pH 9.0/without isopropanol)
R7+ (pH 9.5/with isopropanol)
R7 (pH 9.5/without isopropanol)
R8+ (pH 10.0/with isopropanol)
R8 (pH 10.0/without isopropanol)

### Example 5

Example 5 shows purification of a DNA Ladder from a reaction mixture:
10 μl reaction mixture with Ladder DNA
500 μl binding buffer (1-6) mixed with reaction mixture, after mixing the liquid was pipetted on a Spin Filter.
4 min. centrifugation at 12000 rpm
optionally washing with wash buffer (7-11)
Elution with 20 μl TRIS buffered water
Eluate was completely transferred on a 1.0% agarose gel

### Binding and Wash Buffer:

1) MgCl₂ 50 mM, NaCl 50 mM, isopropanol 50%, pH 9
3) MgCl₂ 90 mM, NaCl 10 mM, isopropanol 50%, pH 9
4) MgCl₂ 10 mM, NaCl 90 mM, isopropanol 50%, pH 9
5) NaCl 100 mM, isopropanol 50%, pH 9
6) MgCl₂ 100 mM, isopropanol 50%, pH 9

### 7) MgCl₂ 50 mM, NaCl 50 mM, pH 9

### 8) MgCl₂ 90 mM, NaCl 10 mM, pH 9

### 9) MgCl₂ 10 mM, NaCl 90 mM, pH 9

### 10) NaCl 100 mM, pH 9

### 11) MgCl₂ 100 mM, pH 9

### Example 5

### Experiment 1

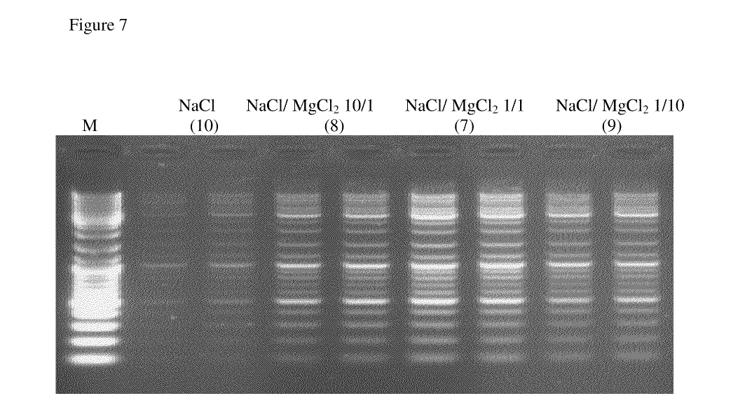

DNA was bound with binding buffer (1) and later on a washing procedure was done with wash buffer 7, 8, 9 and 10 to show the effects of a washing procedure with the wash buffers described in the invention. Results are shown in FIG. 7.

The ideal combination of monovalent to multivalent cations in this experiment is 1:1. The wash buffer is free of any alcoholic component, no drying step after washing is necessary, this reduces the preparation time for up to 20 minutes. Sodium ions alone do not work as a washing buffer.

### Example 5

### Experiment 2

Reducing a binding protocol using the binding buffer as washing buffer in the same step: this leads to a one step purification procedure. The buffers without ethanol described above (7, 8, 9, 10 and 11) were used as binding buffers.

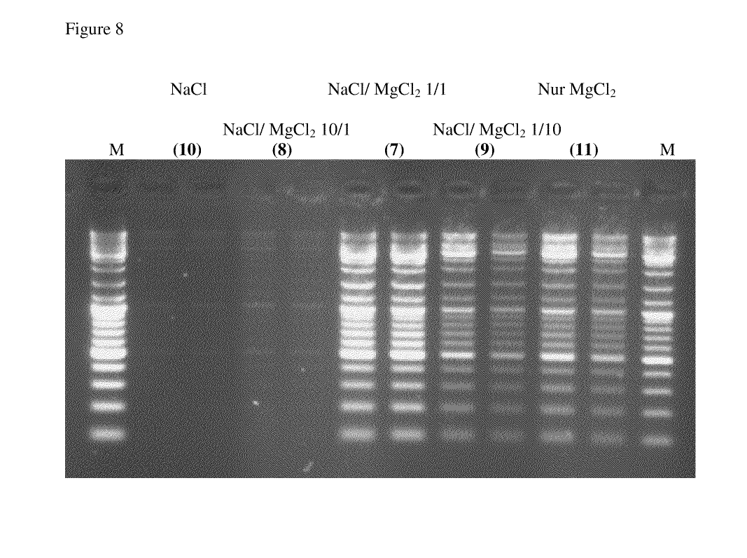

From the left to the right sodium concentration decreases while magnesium concentration increases, in the mid equimolar concentrations are shown in FIG. 8. Sodium alone does not work as a binding agent in this experiment, also magnesium alone shows reduced binding capacity. The best binding results from an equimolar solution of both ions. Shifting concentrations in advantage of one of the ions reduces binding efficiency.

We claim:

1. A method for isolation of nucleic acids from a liquid phase by binding to a solid phase comprising the following steps:
(a) combining a liquid phase containing nucleic acids with additives comprising at least two non-chaotropic cations of different valency, thereby providing a mixture of mono- and multivalent cations;
(b) providing a solid carrier, wherein that solid carrier comprises at least one constituent selected from the group consisting of SiO₂ suspensions, aerosols, magnetized silica particles, cut silic acid, pyrogenous silic acid, magnetic silica particles, glass fiber fleeces, silica membranes or membranes that carry functional groups similar to glass fiber fleeces or silica membranes;
(c) contacting the mixture to the said carrier and binding at least one nucleic acid to said carrier
(d) optionally washing the nucleic acid bound to the carrier by a liquid phase containing at least two non-chaotropic cations of different valency, that finally a mixture of mono- and multivalent cations is combined, without any chaotropic ions or alcohol; and
(e) removing the purified nucleic acid from said carrier by water, buffered water or Tris EDTA-Solution
wherein none of steps (a) through (e) include alcohol, and none of the steps (a) through (e) include chaotropic ions.

2. A method for isolation of nucleic acids from a liquid phase by binding to a solid phase comprising the following steps:
(a) combining a liquid phase containing nucleic acids and alcohol with additives comprising at least two non-chaotropic cations of different valency, thereby providing a mixture of mono- and multivalent cations;
(b) providing a solid carrier, wherein that solid carrier comprises at least one constituent selected from the group consisting of SiO₂ suspensions, aerosols, magnetized silica particles, cut silic acid, pyrogenous silic acid, magnetic silica particles, glass fiber fleeces, silica membranes or membranes that carry functional groups similar to glass fiber fleeces or silica membranes;
(c) contacting the mixture to the said carrier and binding at least one nucleic acid to said carrier
(d) optionally washing the nucleic acid bound to the carrier by a liquid phase containing at least two non-chaotropic cations of different valency, that finally a mixture of mono- and multivalent cations is combined, without any chaotropic ions or alcohol; and
(e) removing the purified nucleic acid from said carrier by water, buffered water or Tris EDTA-Solution.

3. The method of claim 1, wherein the said non-chaotropic mono or multivalent cations are metallic cations.

4. The method of claim 1, wherein the molar ratio of the monovalent cations to the multivalent cations is between 1:9 and 9:1.

5. The method of claim 1, wherein the concentration of each cation in the solution before step (d) is less than 0.5M.

6. The method of claim 2, wherein said alcohol is selected from the group consisting of ethanol, isopropanol, polyethyl glycol, and mixtures of the same.

7. The method of claim 1 wherein the said liquid phase containing nucleic acids further comprises at least one member of the group consisting of detergents and pH fixing substances.

8. The method of claim 1, wherein said non-chaotropic multivalent cations are divalent cations.

9. The method of claim 1, wherein said non-chaotropic multivalent cations are divalent cations.

10. The method of claim 1, wherein said non-chaotropic multivalent cations are selected from the group consisting of Mg²⁺, Ca²⁺, Zn²⁺ and Mn²⁺.

11. The method of claim 1, wherein said non-chaotropic monovalent cations are selected from the group consisting of NH4⁺, Na⁺ and K⁺.

12. The method of claim 1, wherein the pH of the mixture from step c) or f) are adjusted with a TRIS-based buffer.

13. The method of claim 1, wherein the pH of the mixture from step (b) or step (e) is a pH between 8.5 and 9.5.

14. The method of claim 2, wherein the pH of the mixture from step (b) is between pH 5.5 and 9.5.

15. A test kit for isolation of nucleic acids from a liquid phase or lysed solid material comprising:
(a) a binding buffer containing at least two non-chaotropic cations of different valency and
(b) a solid phase.

16. A test kit of claim 15, wherein the binding buffer a) contains at least two non-chaotropic cations of different valency, that finally a mixture of mono- and multivalent cations is combined without any chaotropic ions optionally in combination with an alcohol; and wherein the solid carrier b) comprises at least one constituent selected from the group consisting of SiO₂ suspensions, aerosols, magnetized silica particles, cut silic acid, pyrogenous silic acid, magnetic silica particles, glass fiber fleeces, silica membranes or membranes that carry functional groups similar to glass fiber fleeces or silica membranes; optionally containing a washing buffer compromising at least two non-chaotropic cations of different valency, that finally a mixture of mono- and multivalent cations is combined, without any chaotropic ions or alcohol; and containing an elution buffer from the group of water, buffered water or Tris EDTA-solution.


SEQUENCE LISTING (4 sequences)

SEQ ID NO: 1 - DNA, 190 bp, Artificial Sequence
  PCR reagent
gacgtggacatnatgagagacatcgcctctgggctaataggactacttctaatctgtaagagcagatccctggacaggcgaggaatacaggtattttgtccttgaagtaacctttcagaaattctgagaatttcttctggctagaacatgttaggtctcctggctaaataatggggcatttccttcaaga

SEQ ID NO: 2 - DNA, 193 bp, Artificial Sequence
  PCR reagent
ggagacctagcatgttctagccagaagaaattctcagaatttctgaaaggttacttcaaggacaaaatacctgtattcctcgcctgtccagggatctgctcttacagattagaagtagtcctattagcccagaggcgatgtctctcatgatgtccacgtcactgtagtatggtcttgttaagcactgggcaga

SEQ ID NO: 3 - DNA, 185 bp, Artificial Sequence
  PCR reagent
ggacatgatgagagacatcgcctctgggctaataggactacttctaatctgtnagagcagatccctggacaggcgaggaatacaggtattttgtccttgaagtaacctttcagaaattctgagaatttcttctggctagaacatgttaggtctcctggctaaataatggggcatttccttcaaga

SEQ ID NO: 4 - DNA, 192 bp, Artificial
  PCR reagent
ggagacctaacatgttctagccagaagaaattctcagaatttctgaaaggttacttcaaggacaaaatacctgtattcctcgcctgtccagggatctgctcttacagattagaagtagtcctattagcccagaggcgatgtctctcatgatgtccacgtcactgtagtatggtcttgttaagcactgggcaa